(19) United States
(12) Patent Application Publication
(10) Pub. No.: US 2011/0097410 A1
(43) Pub. Date: Apr. 28, 2011

(54) FORMULATIONS OF PX COMPOUNDS

(76) Inventor: Richard L. Berney, Bethesda, MD (US)

(21) Appl. No.: 12/674,743

(22) PCT Filed: Aug. 21, 2008
(86) PCT No.: PCT/US08/73846

§ 371 Date: Jan. 11, 2011

Related U.S. Application Data
(60) Provisional application No. 60/957,786, filed on Aug. 24, 2007.

(51) Int. Cl. A61K 9/14; A61K 31/225; B82Y 5/00

(52) U.S. Cl. 424/489; 514/547; 977/773; 977/906

(57) ABSTRACT

Nanoparticles of PX compounds in the size range of 10 to 1000 nanometers are incorporated into formulations that are safe for intravenous administration and used to treat disease conditions caused by phospholipase A₂ (PLA₂) such as ischemia-reperfusion injury.

### BACKGROUND OF THE INVENTION

1. Field of the Invention

The invention generally relates to the production of nanoparticles of the PX family of compounds. In particular, the invention provides PX nanoparticles that can be used to prepare formulations that are safe for intravenous administration and used to treat e.g. ischemia-reperfusion injury.

2. Background of the Invention

Compounds of the PX family are known to possess antioxidant activity and to inhibit the enzyme phospholiplase A2 (PLA₂). PX compounds display many salubrious and sanitive properties, and are suggested for use in the treatment of such diverse maladies as central and peripheral neurological inflammation, ischemia-reperfusion injury, malaria, blocking of thrombin-activated platelet activation, psoriasis and atopic dermatitis. Generic depictions of the PX family of compounds are presented in FIGS. 1A-B, and the structures of two representative compounds, PX13 and PX18, are shown in FIGS. 1C-D. The members of this family generally comprise at least two fatty moieties having from 16 to 20 carbon atoms. Further, each fatty moiety has at least one cis-unsaturated double bond, the compounds have no active hydroxyl group, and the compounds may, optionally, have one or more ionizable groups (e.g. carboxyl). U.S. Pat. Nos. 5,659,049; 5,659,055; 5,859,271; 6,020,489; 6,020,510; 6,432,855; and 6,600,059 to Franson and Ottenbrite describe the PX Compounds in detail, and the complete contents of each of the patents are hereby incorporated by reference.

Despite the apparent efficacy of PX compounds in treating various disease conditions, their use is limited due to a problem of extremely low solubility. For example, the solubility of PX-18 in aqueous solutions is approximately 24 μg/ml, and that of PX-13 is only about 5 μg/ml. This very poor solubility generally leads to insufficient bioavailability alter administration, likely because the dissolution velocity is too slow.

According to the Noyes-Whitney equation, which relates the rate of dissolution of solids to the properties of the solid arid the dissolution medium;

$$\frac{d W}{d t} = \frac{D A ( C . - C )}{L}$$

where dW/dt is the rate of dissolution, A is the surface area of the solid, C is the concentration of the solid in the bulk dissolution medium, Cs is the concentration of the solid in the diffusion layer surrounding the solid (saturation solubility), D is the diffusion coefficient, and L is the diffusion layer thickness (Nanosuspensions for the Formulation of Poorly Soluble Drugs, in: Pharmaceutical Emulsions and Suspensions; Nielloud, F. and Marti-Mestres, G., eds.; Marcel Dekker, 383-407, 2000) As can be seen, the dissolution velocity dW/dt is directly proportional to Cs, the saturation solubility. As applied to PX compounds, their solubility is so low that it is not possible to build up sufficiently high concentration in the blood, particularly in short time intervals, for them to be effective for many potential applications. Thus, after oral administration, sufficiently high blood concentrations for bioavailability cannot be achieved. An alternative is intravenous injection. However, too large an injection volume would be required to be feasible, again due to the low solubility of the compounds in water. For example, to administer a dose of only 1 mg/kg body weight, assuming an average patient weight of 80 kg, a volume of 16 liters would be requited to administer an effective dose of PX-13. Thus, prior to this invention, intravenous injection was not possible due to the large volume that would be required.

Alternative parenteral administration routes such as the intramuscular injection of a particle suspension, would result in even lower bioavailability. Such formulations are designed as prolonged release formulations, which means they dissolve slowly to achieve a prolonged blood level. Such a slow dissolution would lead to even longer times to achieve blood levels of the compounds useful for a number of applications, e.g. for the treatment of ischemia-reperfusion, which requires immediate availability the drug. In addition, the concentration of compounds in the blood is continuously reduced due to metabolic degradation of the compounds. Therefore, despite the evidence of beneficial pharmacological actions, these compounds have not heretofore been used to treat patients using intravenous administration.

For many years, micronization has been used as an approach to increase the oral bioavailability of poorly soluble drugs. In this procedure, the drug powder is reduced in size to the low micrometer range, i.e. typically the mean diameter of micronized particles is somewhere between 2 and 10 μm. The increase in surface area leads to an increase is dissolution velocity (according to the Noyes-Whitney equation). However, micronization typically does not lead to a sufficient increase in the dissolution velocity and related oral bioavailability in case of compounds that are even more soluble than PX compounds (Müller and Rasenack, Pharmaceutical Research, 19:12, December 2002).

A second method of increasing the surface area and related dissolution velocity of poorly soluble drugs is nanonization: redaction of size to the nanometer range (i.e. below 1000 nm). The methods used for nanonization are the so-called “bottom-up” technologies and “top-down” technologies. In the bottom-up technologies, one starts from single molecules which are aggregated to form amorphous drug nanoparticles or crystalline drug nanocrystals. Typically, the drug is dissolved in a solvent and the solvent is poured into a non-solvent that is miscible with the solvent. As a result, drug particles precipitate (Sucker et. al., Great Britain Patents 2200048 and 2269536, 1994; Auweter et.al., PCT Application No. PCT/EP2000/003467). However, the bottom-up technologies are problematic in that they are not industrially friendly due to the use of organic solvents.

Top-down, technologies are currently used in the pharmaceutical industry for producing drug nanocrystals. These technologies include ball-milling (Liversidge, et. al., U.S. Pat. No. 5,145,084, 1992); high pressure homogenization (HPH) in water (Müller et. al., U.S. Pat. No. 5,858,410, 1999), or alternatively in water-reduced or water-free media (Müller et. al., PCT Application No. PCT/EP2000/006535). There are also combination technologies in use, for example performing precipitation and subsequent high-energy input in the form of e.g. HPH (James et. al. US patent application No: 20020168402). HPH is accepted by regulatory authorities and HPH plants are currently in use by the pharmaceutical industry for the production of e.g. parenteral emulsions. HPH is an industrially friendly technology since organic solvents are not required. In addition, product contaminations due to erosion from the machines that are used is extremely low, being distinctly below the critical levels (Krause, et. al., International Journal of Pharmaceutics, 196; 2000; 167-172). In this process, the powder is dispersed in a surfactant (e.g. Tween 80) or a stabiliser (e.g. Poloxamer 188) to form a “macrosuspension”. The macrosuspension is then passed through a high pressure homogenizer. Typically, 20 homogenization cycles at a pressure of 1500 bar are applied (Grau, M. J., Kayser, O., Müller, R. H., International Journal of Pharmaceutics, 196; 2000; 155-157).

The ability to reduce particle size of a compound is typically inversely proportional to the water solubility of the compound. Despite a high pressure of 1500 bar and a relatively high number of 20 homogenization cycles, the typical size of nanocrystals obtained with HPH technology is in the range of approximately 300-800 nm for compounds that are more water soluble than PX compounds. However, several commonly used modes of administration (e.g. intravenous administration) require particle sizes of about 250 nm or less. Due to the very low water solubility of PX compounds, it would thus be assumed that they could not be reduced to a size suitable for e.g. intravenous administration. Therefore, even though PX compounds are useful in treating many conditions, their use is restricted to applications that do not require the administration of nanoparticles, and they have not heretofore been used in iv applications to provide sufficiently high bioavailable doses to patients in need, such as those suffering front ischemic-reperfusion events or that will be treated in a manner which exposes them to an ischemic-reperfusion event

### SUMMARY OF THE INVENTION

The present invention provides safe for intravenous formulations containing particles of PX compounds with dimensions in the nanometer range (e.g. below 100 nm) and even low-nanometer range (e.g. about 500 nm or less, or 250 nm or less), where formulations of such particles quickly achieve a sufficient concentration in blood to address, for example, oxidation reperfusion ischemic events, as well as other maladies. According to the invention, PX particles can be made that are of a sufficiently small size so as to form suspensions or colloids in aqueous media, 1) in volumes that are small enough to make intravenous administration feasible; and 2) at concentrations that result in a therapeutically useful level of the PX compound in the blood of a recipient. The suspensions or colloids are stable for more than 3 months. Development of this technology is based on the surprising discovery of the ease with which PX compounds, which are not very water soluble, unpredictably form nanometer sized particles. Even though other similar types of compounds cannot be reduced to nanoparticles by the methods used herein, PX compounds are unpredictably amenable to particle size reduction to the nanometer range.

It is an object of this invention to provide a method of treating or preventing ischemia-reperfusion injury in a mammal in need thereof. The method comprises the step of administering intravenously to the mammal a formulation of PX nanoparticles. The formulation comprises PX nanoparticles of a size in the range of 10 to 1000 nm; and a physiologically compatible carrier (e.g. an aqueous carrier). The PX nanoparticles are suspended in said physiologically compatible carrier. In one embodiment, 95% of the PX nanoparticles are less than 250 nm in size. In another embodiment, 95% of the PX nanoparticles are less than 50 nm in size. In yet another embodiment, the formulation has a volume ranging from 5 to 300 ml, and the PX nanoparticles are present in the formulation at a concentration which will result in a dose to a subject of 20 mg/kg to 80 mg/kg. The PX nanoparticles may include one or more of PX-13 and PX-18.

The invention further provides a formulation of PX compounds safe for intravenous administration. The formulation comprises an aqueous medium and PX nanoparticles of 10-1000 nanometers in size suspended therein. In one embodiment, 95% of the PX nanoparticles are less than 250 nm in size. In another embodiment, 95% of the PX nanoparticles are less than 50 nm in size. In yet another embodiment, the formulation has a volume ranging from 5 to 300 ml, and the PX nanoparticles are present in the formulation at a concentration which will result in a dose to a subject of 20 mg/kg to 80 mg/kg when administered intravenously. The PX nanoparticles may include one or more of PX-13 and PX-18.

### BRIEF DESCRIPTION OF THE DRAWINGS

FIG. 1A-D. Depictions of PX compounds. A, generic depiction of PX compounds that include PX-13; B, generic depiction of PX compounds that include PX-18; C, PX-13; D, PX-18.

FIG. 2. Schematic diagram of correlation between increase in saturation solubility with decreasing particle size (modified after Müller, R. H. and Keck, C. M. (2004) “Challenges and solutions for the delivery of biotech drugs—a review of drug nanocystal technology and lipid nanoparticles.” J Biotechnol 1.13; 1-3:151-70.

FIG. 3. Increase in saturation solubility: comparison, of oleic acid to water.

FIG. 4. Cardioprotective effect of PX-18 on infarct size.

FIG. 5. Experimental protocol for testing PX-18 influence on basal bleed flow and various dilating stimuli.

FIG. 6. Effect of iv PX-18 on basal and hypercapnia-induced (endothelium and cyclooxygenase(COX)-dependent) pial arteriolar dilation.

FIG. 7. Effect of topical PX-18 on PACAP induced (non-endothelium but COX-dependent) pial arteriolar dilation.

FIG. 8. Experimental protocol tor testing PX-18 influence on ischemia-reperfusion injury.

FIG. 9. Effect of PX-18 on ischemia-induced impaired vasodilation due to bradykinin.

FIG. 10. Effect of PX-18 on ischemia-induced impaired vasodilation due to N-methyl-D-aspartic acid (NMDA).

FIG. 11. Effect of IV vs topical treatment with PX-18 on impaired hypercapnic vasodilation after global ischemia.

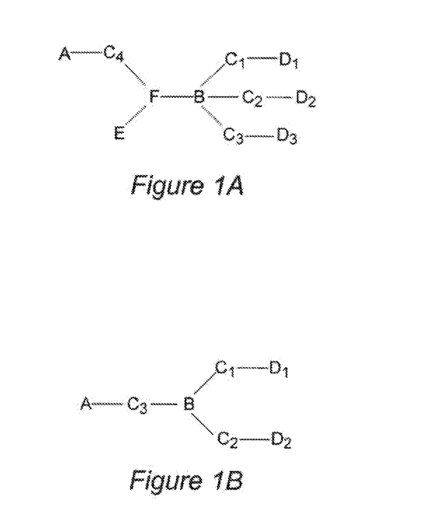

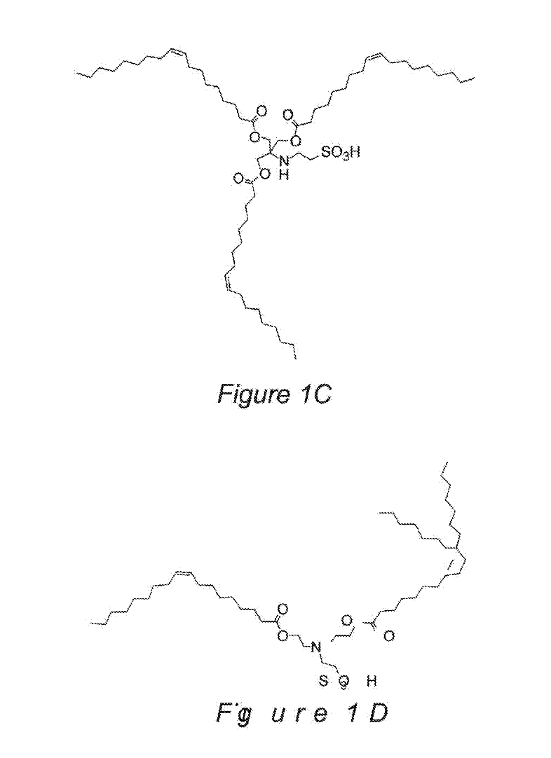

### DETAILED DESCRIPTION

The present invention is based on the discovery that, contrary to expectations based on their lack of water-solubility, nanoparticles of PX compounds ranging in size from about 10 to about 1000 nm may be prepared in a facile manner. Surprisingly it has been found that only one homogenization cycle at 1500 bar is sufficient to produce, for example, PX-18 particles less than 800 nm in size. The application of 20 homogenization cycles at 1500 bar led to PX-18 nanoparticles of a size in the very low nanometer range, i.e. 40 nm. Such particles are suitable for intravenous administration.

The generic formula for the PX compound family that includes PX-13 is presented in Formula 1:

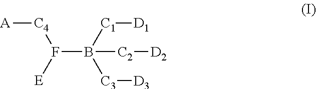

In this formula, A comprises H, OH, a sugar moiety, an ether, an ester, an amide or NH₂, or an acid or salt thereof. Some of the preferred, acids that may be substituted for A include but are not limited to COOH, SO₃H or PO₃H. B is a connecting group selected from the group consisting of C, —(CH₂)ₙC—, N⁺, and (CH₂)ₙ N⁺ wherein n is an integer from 1 to 24,and the —(CH₂)ₙ chain may be functionalized or non-functionalized. C₁, C₂, C₃ and C₄ are connecting groups selected from the group —(CH₂)ₙ—where n is an integer from 1 to 24, wherein the —(CH₂)ₙ—chain may be functionalized or nonfunctionalized. C₁, C₂, C₃ and C₄ may also be selected from the group consisting of poly(ethylene oxide) of the formula (CH₂-CH₂—O)_{y} wherein y is an integer from 1 to 12. C₁, C₂, C₃ and C₄ may be the same or different. D₁, D₂, and D₃ are fatty chains consisting of fatty acid esters of the form CH₃(CH₂)ₙCOO or fatty acid amides of the form CH₃(CH₂)₃CONH wherein n is an integer from 0 to 32. At least one of the fatty chains is unsaturated at one or more positions, and D₁, D₂ and D₃ may be the same or different with respect to length and degree of unsaturation. D₁, D₂ or D₃ may also be H provided no more than one of D₁, D₂ and D₃ is H in anyone compound. In the generic formula listed above, the fatty acid chains of the molecule may be comprised of (CH₂)ₙ wherein n is an integer from 1 to 32. These fatty acid chains may each be unsaturated at one or more sites and may be of different lengths from 1 to 32 carbon atoms. E is H, or is the same as A-C₄, or is a fatty acid amide of the form CO(CH₂)ₙCH₃ or CO(CH₂)ₙCOOH where n is an integer from 0 to 24. The alky (CH₂)ₙ chain may be functionalized or nonfunctionalized. F is selected from tire group consisting of N, NR, P, P═O, CH or CR, wherein R is an alky chain of 1 to 6 carbons which may be functionalized or non-functionalized.

In one embodiment, PX compounds of generic Formula 1 have the following structure:

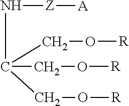

wherein the R groups may be the sane or different and each R is a fatty moiety as set forth below,

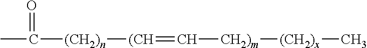

wherein n is an integer from 1 to 18, m is an integer from 1 to 4,and x is an integer from 0 to 12. In this form of the present invention Z represents a C₁ to C₅ aliphatic moiety, which may be functionalized or non-functionalized, and A represents an organic acid moiety or salt form thereof or any organic acid radical. It is to be understood that the acidic groups and the NH groups in the generic structure described above may be present in ionized form. Some of the preferred acid groups that may be substituted for A include but are not limited to COOH, SO₃H or PO₃H. PX-13 belongs to this class of compound.

A generic representation of the PX compound family that includes PX-18 is depicted in Formula 2:

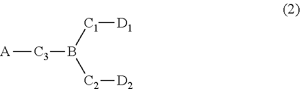

In this formula, A comprises H, OH, a sugar moiety, an ether, an ester, an amide or NH₂, or an acid group or salt thereof. Some of the preferred acids include COOH, SO₃H, and PO₃H. B is N, NR, P, P═O, CH or CR wherein R is an alkyl chain of 1 to 6 carbons which may be functionalized or non-functionalized. C₁, C₂ and C₃ are connecting groups selected from the group consisting of —(CH₂)ₙ-wherein n is an integer from 1 to 24,and the —(CH₂)ₙ˜chain may be functionalized or non-functionalized. C₁, C₂ and C₃ may also be selected from the group consisting of poly(ethylene oxide) of the formula (CH₂CH₂—O)y wherein y is an integer from 1 to 12. C₁, C₂, and C₃ may be the same or different. D₁ and D₂ are fatty acid chains consisting of fatty acid esters of the form CH₃ (CH₂)ₙ COO or fatty acid amides of the form CH₃(CH₂)ₙCONH wherein n is an integer from 1 to 32. At least one of the fatty chains is unsaturated at one or more positions, and D₁ and D₂ may be the same or different with respect to length and degree of unsaturation. In the generic formula listed above, the fatty acid chains of the molecule may be comprised of (CH₂)ₙ wherein n is an integer from 1 to 32. These fatty acid chains may each be unsaturated at one or more sites and may be of different lengths from 1 to 32 carbon atoms.

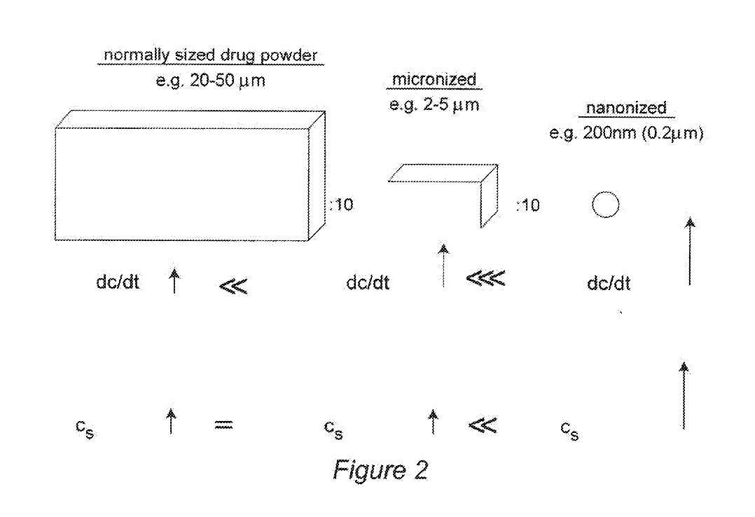

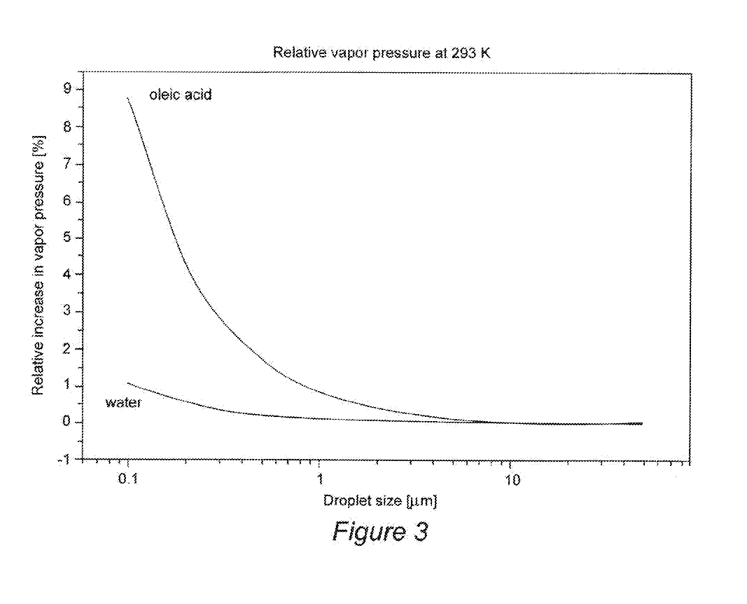

Size reduction of particles of such PX compounds to 1000 nm and below promotes rapid suspension of the PX material. Without being bound by theory, this is likely due to the fact that, with decreasing particle size, both the surface area increase and the saturation solubility (Cs) contribute to an increase in dissolution velocity, dc/dt. In general, saturation solubility increases not linearly but exponentially with decreasing particle size, having a pronounced effect below about 500 nm, an even more pronounced effect below 100-200 nm, and being highest in the range of 50 nm and below. FIG. 2 (reproduced from Müller, R. H, and Keck, C. M., 2004, “Challenges and solutions, for the delivery of biotech drugs—a review of drug nanocrystal technology and lipid nanoparticles,” J Biotechnol 113:1-3; 151-70) shows the basic relationship between reduction in particle size and the concomitant increase in saturation solubility. Increases in saturation solubility may also be predicted on the basis of corresponding increases, in vapour pressure. FIG. 3 shows that, using the model compounds water and oleic acid, the increase in saturation solubility is much higher for oleic acid compared to water. This means that for such compounds as oleic acid, it is not necessary to go to nanoparticles below 100 nm to achieve a similar increase in saturation solubility. For compounds showing a strong increase in saturation solubility with size reduction, nanoparticles in the size range of 500-1000 nm can be sufficient to prepare formulations that are suitable for intravenous administration. However, this is not the case for PX compounds, for which preferable particles sizes are less than about 500 nm, or even less than about 250 nm.

According to the invention, a preferred PX nanoparticle size is 1000 nm or less, or more preferably about 500 nm or less, or even more preferably about 250 nm or less, and most preferably about 100 nm or less, or even 50 nm, or less. The lower limit of PX nanoparticle is about 10 nm. By nanoparticle “size” or “size range” we mean that the average longest dimension of the nanoparticles in a collection or preparation of nanoparticles falls within the range. Those of skill, in the art will recognize that the “longest dimension” of a particle will depend on the shape of the particle. For example, for particles that are roughly or substantially spheroid, the longest dimension will be a diameter of the particle. For other particles (e.g. crystals that may have, for example, angular shapes) the longest dimension may be e.g. a diagonal, a side, etc. Those of skill in the art will also recognize that in a given preparation of nanoparticles, the sizes and shapes of the particles may vary. However, according to the present invention, the average of a longest dimension of the particles in the preparation is in the range of from about 10 to about 1000 nm, and preferably from about 10 to about 500 nm, more preferably from about 10 to about 250 nm, and most preferably from about 10 to about 100 nm, or less (e.g., 50 nm or less). In particular, particles in the size range of less than about 250 nm, or less than about 100 nm, and preferably less than 50 nm, are especially suitable for use in formulations for intravenous administration.

The nanoparticles of PX compounds may be in any of several solid states known to those of skill in tire art, and their precise form will depend e.g. on the method of synthesis of the compound, on its handling after synthesis, etc. For example, the nanoparticles may be in a crystalline state or in an amorphous state, micelles, or liposomes or a mixture of one or more of these.

Reduction of PX compounds to nanoparticles may be earned out by any method known to those of skill in the art, examples of which include but are not limited to high pressure homogenization, pearl/ball milling, precipitation, and combinations thereof. In particular, a combination of precipitation and high pressure homogenization has been found to be efficacious in producing PX nanoparticles. However, based on the ease of size reduction of the PX compounds using these methods, other technologies can likely also be applied successfully. The intrinsic property of the PX compound family of ease of size reduction leads to very short processing times of all production methods associated with an input of energy. Further, adjustments in the size of the nanoparticles may be made e.g. by increasing or decreasing the parameters or conditions under which a nanonization procedure is carried out, and may also depend on the particular PX compound that is being nano-sized. For example, one homogenization cycle at 1500 bar produces 400-800 nm-sized particles of PX-18 and 20 homogenization cycles produces approximately 40 nm-sized particles of PX-18. One homogenization cycle at 1500 bar produces 400-900 nm sized particles of PX-13, and applying 20 homogenization cycles produces approximately 250 nm sized particles.

In a preferred embodiment, the invention provides nanoparticle formulations of PX compounds that are safe for intravenous administration. For intravenous formulations, which require a volume of about 50 to 300 ml or less for administration, nanoparticles of 250 nm or less, and preferably of 100 nm or less, or even 50 or 40 nm or less, may be preferred. However, nanoparticle formulations of PX compounds may also be used for other types of administration, which may or may not be sensitive to particle size. Those of skill in the art will recognize that it is generally beneficial to create low-volume formulations for any type of administration, and that the increased absorption of the PX compounds may prove advantageous for all types of administration.

The invention provides formulations of PX nanoparticles for therapeutic applications, particularly for therapeutic applications which require or are typically treated by intravenous administration. Importantly, by decreasing the size of the particles, formulations may be made of sufficiently high concentrations so as to cause or bring about a beneficial therapeutic effect for the recipient. By using nanoparticles as described herein, blood levels that are sufficient to treat symptoms of conditions that can be ameliorated by PX compounds can be safely achieved. The bioavailability of the administered nanoparticles is high enough to result in a beneficial effect of lessening or eliminating symptoms of disease.

The invention further provides methods of treating disease conditions associated with the activity of the enzyme PLA₂, particularly sPLA₂. The methods generally involve the administration (in particular, IV administration) of a therapeutically effective amount of the PX nanoparticles of the invention. A “therapeutically effective amount” or a “therapeutic effective amount” of a PX compound is an amount that alleviates, totally or partially, a pathophysiological effect. A therapeutically effective amount or a therapeutic effective amount can also be an amount that is given prophylactically thereby inhibiting a pathophysiological effect (e.g. inflammation). The amount will depend upon, for example, subject size, gender, age, the magnitude of the condition being treated, and various genetic or non-genetic factors associated individual pharmacokinetic or pharmacodynamic properties of the administered compound. For a given subject in need thereof, a therapeutically effective amount can be determined by those of ordinary skill in the art and by methods known to those of ordinary skill in the art. As discussed herein, for PX compounds in the form of nanoparticles of a size that is less than about 1000 nm, preferably less than about 500 nm, more preferably less than about 250, or even 100, or even 50 nm, that amount will generally be in the range of from about 10 to about 100 mg/kg, and preferably from about 20 to about 80 mg/kg.

In some embodiments, the administration of PX nanoparticles exerts its therapeutic effects by inhibiting the activity of PLA₂ enzymes. To “inhibit” the activity of PLA₂ as used herein has the generally understood meaning, e.g. to decrease, attenuate, restrict, reduce, repress, lessen, slow, lower, etc. Such inhibition is generally characterized by a decrease in the activity that is inhibited of at least about 10%, 20%, 30%, 40%, 50%,60%, 70% 80%, or 90% or more. Preferably, the decrease is at least about 50%, more preferably at least about 75%, and most preferably about 90% or more, hi some cases, the activity may be inhibited 100%, i.e. the activity may be entirely abolished.

In some embodiments the nano-sized PX compounds administered as liquids (e.g. suspensions or colloids) with a physiologically or pharmacologically suitable (compatible) carrier. The preparation of such compositions is well known to those of skill in the art. Typically, such compositions are prepared either as liquid suspensions or colloids. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. The final amount of active agent(s) in the formulations may vary. However, the amount of PX compound in such compositions will generally be in the range of about 1-99%, Further, more than one PX compound may be included in a formulation and administered IV to a patient in particular, such formulations may include one or more surfactants and/or emulsifiers, examples of which include but are not limited to Tween® 80, Tween® 20 etc. In addition, the formulations may include one or more stabilizers, examples of which include but are not limited to Poloxamer 188, polyvinylpyrrolidone,, egg lecithin, soy lecithin, etc. In one embodiment, the invention contemplates compositions particularly for intravenous (IV) administration of PX compounds, and the compositions of the invention may contain any additional ingredients required to provide the composition in a form suitable for IV administration.

The PX nanoparticles of the invention may also be administered in other forms. Other forms for administration of the PX nanoparticles include but are not limited to, for example, various solid forms suitable for suspension in liquids prior to administration, or solid forms that are administered without suspension. Examples of this aspect of the invention include various pills, powders, gels, pastes, ointments, etc, which may serve as a vehicle for delivery of PX nanoparticles. Those of skill in the art are familiar with the production of such drug vehicles and formulations, and all such that are known are intended to be encompassed by the present invention.

The precise form for delivering the PX nanoparticles may depend, for example, on the condition that is being treated. In particular, for the treatment of ischemia-reperfusion injury, intravenous formulations may be used. However, the treatment of other conditions may require other formulations. For example, topical preparations such as ointments or salves may be used to treat skin conditions; injectable liquid forms may be used when subcutaneous delivery of the compound is advisable; various aerosol formulations may be used for delivery to the nasal passages and lungs; liquid drops may be used for intraocular administration, etc. Those of skill in the art are familiar with such distinctions and with methods for preparing suitable formulations.

The PX nanoparticle compositions of the invention may be used to treat a variety of disease conditions, especially those associated with PLA₂ activity. In a preferred embodiment, intravenous formulations of PX compounds are used to treat ischemia-reperfusion injury. However, this does not exclude the treatment of other conditions, including but are not limited to: central and peripheral neurological inflammation, malaria, psoriasis, atopic dermatitis, etc. The formulations may also be used as bronchodilators, bladder relaxants, islet cell protectors, anti-epileptics, as promoters of hair growth, and for the treatment of male erectile dysfunction. In addition, administration of the formulations may be used advantageously to block thrombin-activated platelet activation.

In some instances, the PX nanoparticles may be administered prophylactically before any disease symptoms are present. This may be the case, e.g. for cardiac conditions, in which a patient may be known to be at risk for an infarction land/or ischemic/reperfusion injury, but has not yet undergone such a trauma. For example, a patient for which surgery is planned may be at risk, and the prophylactic administration of PX nanoparticles may be warranted. In other instances, the PX nanoparticles may be administered after disease symptoms are noted, particularly if the condition does not involve advanced warnings signs. This may be the case, for example, with skin disorders such as psoriasis.

In some embodiments of the invention the patient or subject is a mammal, frequently a primate, and in preferred embodiments of the invention the mammal is a human, although this need not be the case. Veterinary applications of this technology are also contemplated, as are applications that involve laboratory research, i.e. the PX nanoparticles of the invention may be used as research agents.

In the Examples section below. Example 1 illustrates the successful use of PX compounds to treat ischemia-reperfusion injury using methods of administration other than IV. Examples 2-3 illustrate that PX compounds can readily be reduced to nanoparticle sizes that can be used in IV formulations. Examples 4-5 show that such IV formulations can be used to successfully treat ischemia-reperfusion injury.

### EXAMPLES

### Example 1

### Protective Effect of PX-18 during Ischemia-Reperfusion

The protective effect of PX-18 during ischemia-reperfusion in the heart was assessed. Since opening of mitochondrial KATP channels are essential components of ischemic tolerance induced by preconditioning in heart, the role of these channels in PX-18-induced cardioprotection was also examined. Briefly, 180 mg of PX-18 was put into a small homogenizing tube containing 3 ml of ethylene glycol in a homogenizing tube and warming it to about 50° C. Several bursts of sonication (about 10 seconds) were applied in a nitrogen gas bath to minimize oxidation, followed by homogenization. This procedure was repeated 4-5 times until a uniform cloudy proportion that was easily injected through a needle was obtained. Procedures of this type generate PX-18 particles of a size in the micrometer range. Rabbits were treated with the sonicated preparation, of PX-18 (60 mg/kg ip). 30 minutes later the hearts were subjected to 30 minutes of regional ischemia followed by 3 hours of reperfusion. Mitochondrial KATP channel blocker 5-hydroxydecanoate (5-HD, 5 mg/kg iv) was given 10 min before ischemia/reperfusion. Infarct size was measured by computer morphometry of tetrazolium stained sections.

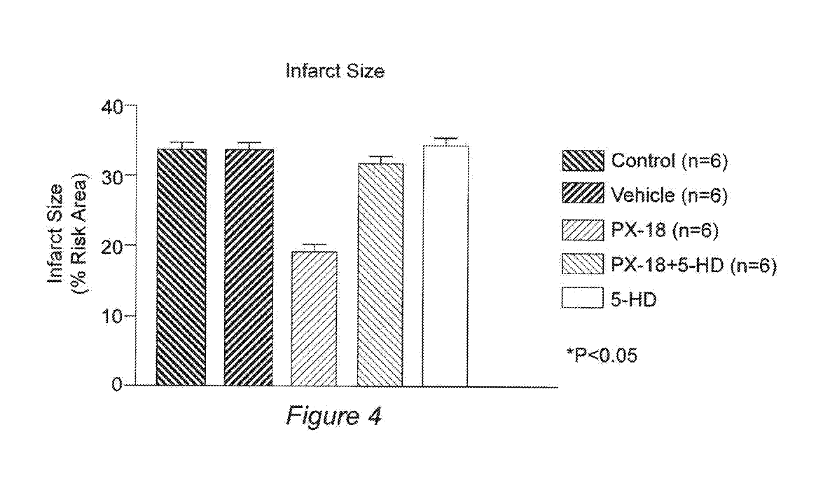

The results, which are presented in FIG. 4, showed that the infarct size (% risk area, means+SE) in PX-18 treated rabbits was reduced from 33.8=1.3% for vehicle (ethylene glycol) to 193=1.0% (43% reduction, P<0.05). HD blocked the cardioprotective effect of PX-18 as indicated by the increase in infarct size to 31.9=1.2% (P<0.05 vs. PX-18). Animals treated with 5-HD alone had an infarct size of 34.9=1.1 % which was not different from the saline treated control (33.7=1.3%) or vehicle treated (33.8=1.4) ischemic/reperfused animals (P>0.05). The risk areas were not different among the various groups.

A similar experiment was attempted post ischemia using the same sonicated formulation of PX-18 administered intravenously, but the rabbits did not survive.

The example shows that although PX-18 induces pharmacological preconditioning and is efficacious in significantly, reducing infarct size when administered ip, preparations of PX-18 in which the particle size is in the micrometer range are not suitable for iv administration.

### Example 2

### Nanonization of PX-18 and PX-13

In order to prepare PX particles of a nanometer size range that could be safely administered intravenously, the following experiments were carried out.

PX-18 powder was dispersed in a 1% Tween 80 carrier. The composition of the formulation was 1% PX-18, 1% Tween 80 and 98% water. The obtained macrosuspension was passed through a micron LAB 40 homogenizer (APV Homogenizer Systems, Unna/Germany) at 1500 bar, applying one homogenization cycle. The particle size was determined by photon correlation spectroscopy (PCS, Malvern Zetasizer Nano ZS, Malvern Instruments, United Kingdom), The mean particle size (z-average) was 745 nm.

A second macrosuspension of PX-18 was prepared as described above, and subsequently homogenized. The homogenization parameters were 1500 bar and 20 homogenization cycles. Surprisingly, this resulted in a mean PCS diameter of 41 nm.

A second PX-compound, PX-13 was also investigated and. showed identical properties regarding the ease of size reduction. After 20 homogenization cycles a size of 33 nm was attained. Such size reductions of PX compounds have not previously been obtained with high pressure homogenization.

A suspension of 1% PX-13 was prepared in an aqueous preparation of 2% Planta Care 2000. After homogenization applying 20 cycles at 1500 bar, a PCS mean diameter of 33 nm was obtained.

This example shows that members of the PX family of compounds unexpectedly and readily undergo size reduction to particles of less than 50 nm.

### Example 3

### Dissolution Velocity of PX Nanoparticles

The increase in dissolution velocity was investigated by comparing nanoparticles versus PX-18 nanoparticles. In order to increase oral bioavailability, and also to achieve high levels of PX-18 In the blood after intravenous injection, it is essential that the PX-18 particles dissolve quickly. For example, if slow dissolution of intravenously injected particles occurs, the particles will be recognized as being foreign by the macrophages of the body and will be taken up by the macrophages of the liver very quickly (e.g. uptake of up to 90% of injected dose within 5 minutes; Müller, R. H., Colloidal Carriers for Controlled Drug Delivery and Targeting—Modification, Characterization and in vivo Distribution, Wissenschaftliche Verlagsgesellschaft Stuttgart, CRC Press-Boca Raton, 379 S., 1991, p 337-345). This Example shows that suspension of the PX nanoparticles of the invention, as described herein is very fast. After just 5 minutes 91% of the nanoparticles were suspended compared to just 8.4% of the powder containing μm-sized particles.

1 ml of macrosuspension (PX powder, dispersed in water) and 1 ml of PX-18 nanosuspension (diameter 40 nm) was added to 9 ml of de-mineralised water and stirred with a magnetic stirrer for 5 minutes. Each 10 ml sample was then filtered to remove non-suspended PX-18, and the amount of suspended PX-18 was determined by gravimetry. While the concentration of PX-18 in both suspensions was 1% (w/w), about 91% of the nanoparticles had become suspended within 5 minutes compared to just 8.4% of the macrosuspension.

To farther assess the importance of nanoparticle size on the dissolution velocity of PX-18, the dissolution velocity of PX-18 nanoparticles with a size of 40 nm was compared to that of PX-18 nanoparticles with a size of 200 nm. The analysis was performed as described above. After 5 minutes, about 91% of the 40 nm particles were suspended, whereas die larger 200 nm nanoparticles were suspended to about 41%.

This example shows that deceasing the size of particles of PX compounds dramatically increases their ability to be suspended in aqueous media, making possible the production of formulations suitable for intravenous administration.

### Example 4

### Protective Effect of Nanoparticulate PX-18 during Ischemia-Reperfusion

Common carotid arteries of Gerbils (6 gerbils per group) were occluded for 5 minutes to induce ischemia. Thereafter, a nanoparticle formulation of PX-18 (1% formulation, particles size of 40-50 nanometers) was administered subcutaneously 5 min, 24 hr and 48 hr after ischemia. After surgery, the gerbils were returned to their cages where, upon awakening, they were able to resume their normal locomotor functions. They were sacrificed on day 4. The hippocampus was sectioned and the number of alive neurons was assessed. In the ischemic animals, sPLA₂ unregulated within 24 hr after ischemia and most neurons were dead by 4 days. PX-18 reduced neuronal damage in a dose dependent manner, providing approximately 30% protection at a dose of 30 mg/kg and 45% protection at a dose of 60 mg/kg,

This Examples show that PX-18 nanoparticles in the nanometer size range protect neurons against ischemic injury when administered ip.

### Example 5

### Influence of Intravenous PX-18 Nanoparticles on the Hemodynamics of Circulation, Cerebrovascular Dilatory Responses and Ischemia/Reperfusion (IR)-Induced Cerebrovascular Dysfunction

PX-18, a specific inhibitor of sPLA₂, is a fatty moiety feat is soluble in methylene chloride, chloroform and ethyl acetate. It has limited solubility in dimethylsulfoxide (DMSO). it is insoluble in acetone and methanol. Its solubility in water is typically very low. However, its ability to be formulated into aqueous solutions is significantly improved by nanonization, as shown in Examples 2 and 3 above.

The ability of PX-18 nanoparticles to influence 1) basal and induced arterial dilation; and 2) ischemia-induced impaired vasodilation (due to bradykinin, N-methyl-D-aspartic acid (NMDA) and ischemia-reperfusion) was studied in newborn piglets (1-2-day old, n=44). The piglets were anesthetized, ventilated, and equipped with arterial and venous catheters and a closed cranial window. Xanoparticulate PX-18 was administered iv at a concentration of 6 mg/kg.

The results showed that pial arterioles were significantly dilated (16±5%, Mean±SEM) by a nanoparticle formulation of PX-18 (10⁻⁴M) administered topically onto the brain surface, but not by lower concentrations (10⁻⁸-10⁻⁵M). An intravenous (iv) bolus of a nanoparticle formulation of PX-18 (6 mg/kg) transiently decreased mean arterial blood pressure (MABP) from 62±7 mmHg to 40±5 mmHg and dilated, pial arterioles (32±1%), However, both normalized after 4-6 minutes (not shown).

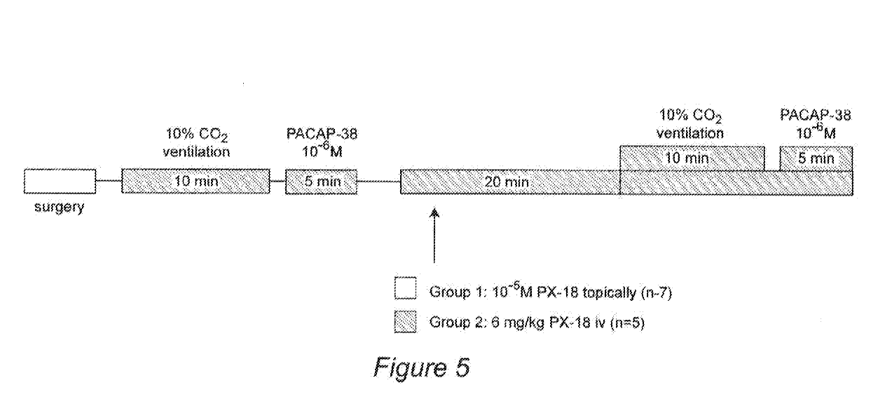

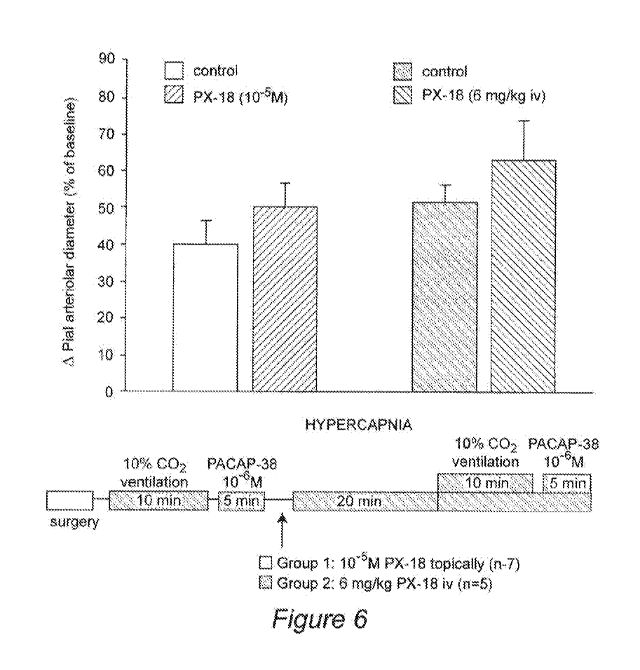

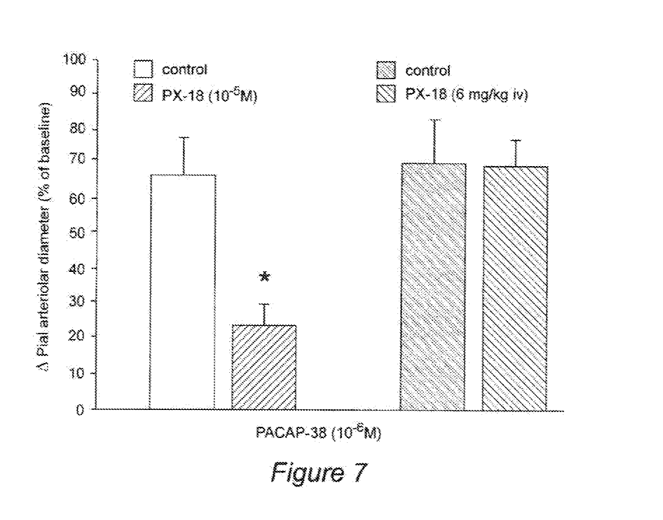

The protocol for testing die effects of PX-18 on basal and induced arterial dilation is presented in FIG. 5. Results showed that the effects were dependent on the agent that induced dilation. As shown in FIG. 6, intravenous PX-18 did not influence basal and endothelium, and cyclooxygenase (COX) dependent hypercapnia-induced pial arterial dilation. However, as shown in FIG. 7, topical PX-18 impaired non-endothelium but COX-dependent pial arteriolar dilation, PACAP

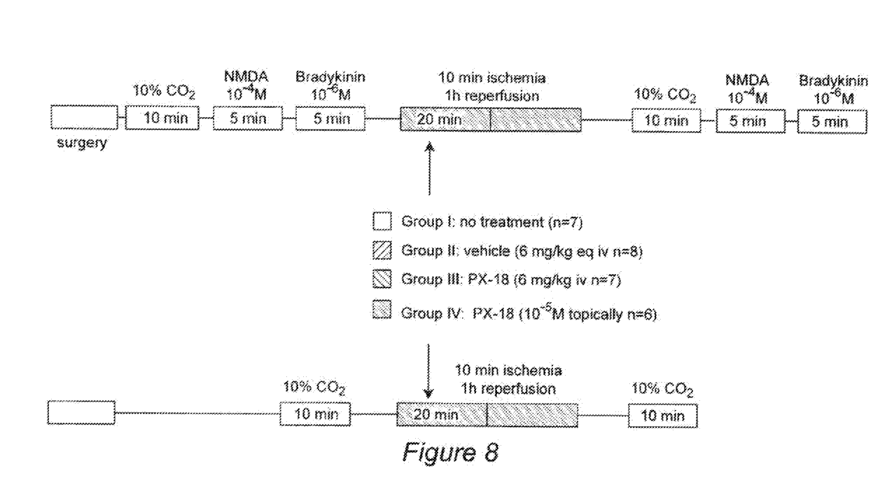

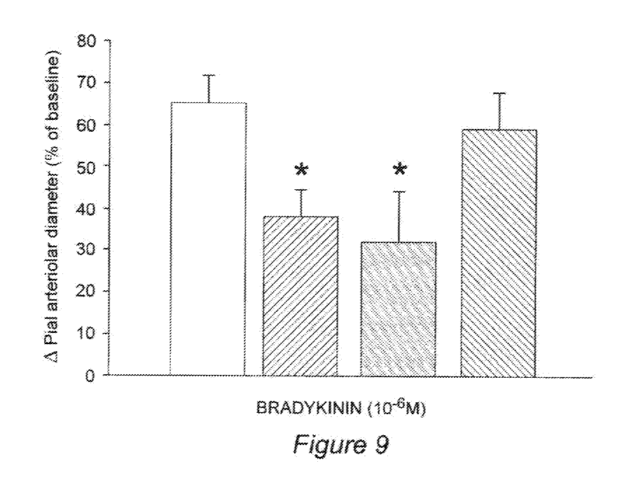

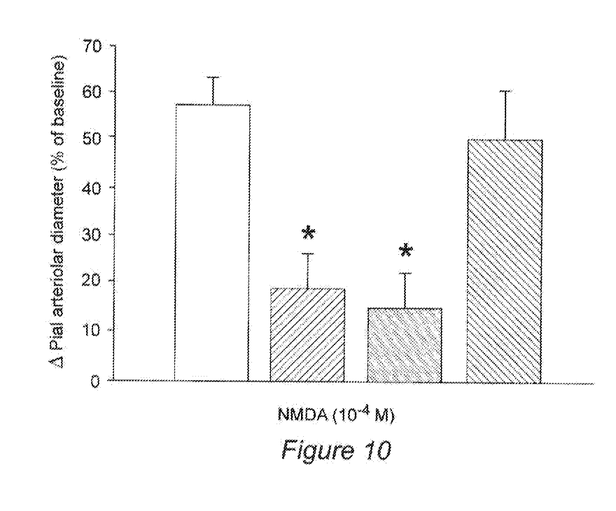

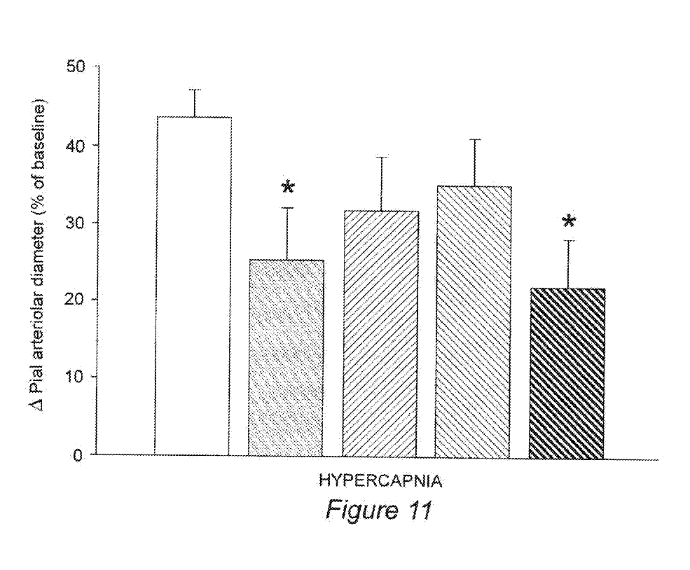

The protocol for testing the effect of ischemia-induced impaired vasodilation is preseted in FIG. 8. The results showed that iv administered PX-18 counteracts the ischemia-induced impaired vasodilation caused by both bradykinin (10⁻⁸M, FIG. 9) and N-methyl-D-aspartic acid (NMDA, 10⁻⁴M, FIG. 10). In addition, intravenous but not topical treatment with PX-18 restored impaired hypercapnic vasodilation after global ischemia (FIG. 11).

These results indicate that PX-18 is highly effective in protecting against I/R-induced cerebrovascular injury when administered iv. However, as a matter of safe administration, the PX-18 dosage needs to be carefully regulated so that concentrations reaching the brain surface do not exceed 10⁻⁵ M. Since PX-18 is highly insoluble, it is critical that a homogenous suspension not have particles of greater than 100 nanometers in its longest dimension.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method of treating or preventing ischemia-reperfusion injury in a mammal in need thereof, comprising the step of
administering intravenously to said mammal, a formulation of PX nanoparticles, said formulation comprising
PX nanoparticles of a size in the range of 10 to 1000 nm; and
a physiologically compatible carrier, said PX nanoparticles being suspended in said physiologically compatible carrier.

2. The method of claim 1 wherein 95% of said PX nanoparticles are less than 250 nm in size.

3. The method of claim 1 wherein 95% of said PX nanoparticles are less than 50 nm in size.

4. The method of claim 1 wherein said formulation has a volume ranging from 5 to 300 ml, and wherein said PX nanoparticles are present in said formulation at a concentration which will result in a dose to a subject of 20 mg/kg to 80 mg/kg.

5. The method of claim 1 wherein said PX nanoparticles include one or more of PX-13 and PX-18.

6. A safe for intravenous administration formulation of PX compounds, comprising:
an aqueous medium; and
PX nanoparticles of 10-1000 nanometers in size suspended in said aqueous medium.

7. The safe for intravenous administration formulation of PX compounds recited in claim 6 wherein 95% of said PX nanoparticles are less than 250 nm in size.

8. The safe for intravenous administration formulation of PX compounds recited in claim 6 wherein 95% of said PX nanoparticles are less than 50 nm in size.

9. The safe for intravenous administration formulation of PX compounds recited in claim 6 wherein said aqueous medium has a volume ranging from 5 to 300 ml, and wherein said PX particles are present in said aqueous medium at a concentration which will result in a dose to a subject of 20 Mg/kg to 80 mg/kg when administered intravenously.

10. The safe for intravenous administration formulation of PX compounds of claim 1 wherein said PX nanoparticles include one or more of PX-13 and PX-18.
